(19) United States
(12) Patent Application Publication
(10) Pub. No.: US 2012/0016296 A1
(43) Pub. Date: Jan. 19, 2012

(54) AUTOINJECTOR WITH MIXING MEANS

(75) Inventor: Cleathero Ian Charles, Leicestershire (GB)

(73) Assignee: The Medical House Limited

(21) Appl. No.: 13/059,900

(22) PCT Filed: Aug. 5, 2009
(86) PCT No.: PCT/GB09/50984

§ 371 Date: Jul. 14, 2011

(30) Foreign Application Priority Data
Aug. 18, 2008 (GB) 0814985.8

(51) Int. Cl. A61M 5/31

(52) U.S. Cl. 604/87

(57) ABSTRACT

An autoinjector comprising:
a first chamber for containing a dry component of a medicament;
a second chamber for containing a wet component of a medicament;
an axially-slidable stopper intermediate said first and second chambers;
an injection needle in fluid communication with said first chamber; and
a transfer needle initially disposed axially forward of said stopper, the transfer needle having a longitudinal axial bore therethrough, a closed forward or proximal end, an open rear or distal end and a radial aperture intermediate said proximal and distal ends and in fluid communication with said longitudinal axial bore;
wherein said transfer needle is capable of penetrating said stopper to establish fluid communication between said first and second chambers via said bore and radial aperture to enable said wet component to mix with said dry component and wherein said injection needle is capable of delivering the mixed dry and wet components of the medicament to an injection site.

This invention relates to the field of autoinjectors for the administration of liquid medication.

### BACKGROUND

An autoinjector is an automatic injection device designed to facilitate automated delivery of a dose of medicament to a patient through a hypodermic needle, the injection usually being administered by the patient themselves. The injection can be delivered subcutaneously or intra-muscularly, depending upon the properties of the autoinjector. An autoinjector works by delivering an injection automatically upon actuation by the patient, for example pressing a button, moving a lever or part of a housing etc. This is in contrast to a conventional manual syringe where the patient himself needs to directly depress a plunger into a barrel containing medicament in order to effect the injection. The terms “autoinjector” and “injection device” are used interchangeably in the following description.

Some types of medication for delivery by an autoinjector are provided and stored in a two-part form, having a solid component and a liquid component, for example as a powdered medicament and a liquid solvent. The powdered medicament may comprise a ground-down or milled solid medicament or may be a powder prepared by a lyophilisation process for example. The liquid part may be a second medicament, rather than a solvent or diluent. Such two-part formulations are well known and sometimes referred to as “wet-dry” formulations. Immediately before the medicament is delivered by injection into the patient, the wet and dry components of the formulation are mixed together so that a combined medicament is delivered by the autoinjector.

During storage and before use of the autoinjector, it is essential that the wet and dry components of the formulation are kept separate from one another within the autoinjector. When it is desired to deliver an injection, the wet and dry components need to meet together quickly and effectively before being expelled from the injection device.

A prior art autoinjector is described in EP0361668 (Medimech Limited) in which an autoinjector has at least two chambers containing different ingredients of a medicament separated by an impermeable membrane. When it is desired to deliver an injection, a lance moves to cut or pierce the membrane allowing the ingredients to mix immediately before a plunger drives a needle out of the body of the injector to discharge the medicament through the needle.

Another autoinjector is described in WO02/49691 (Gillespie) in which a spring-operated plunger forces pressurised liquid from a first chamber causing a releasable seal between the first and a second chamber to disengage so that liquid can flow through the second chamber dissolving any dry medicament therein.

Another autoinjector is described in EP1709984 (Meridian Medical Technologies, Inc) in which the liquid is pressurised upon actuation of the device such that a fluid passageway between first and second chambers is opened in order that the wet and dry components can meet. In an alternative embodiment, a seal between the first and second chambers is punctured by a spike, allowing their respective contents to mix.

In all three of the above prior art devices, mixing of the medicament is effected almost simultaneously with expulsion of the medicament from the device, the wet component being “flushed” through the dry component on its way out of the autoinjector. This has the advantage of reducing the total time between the user actuating the device and delivery of the injection.

It is an object of the present invention to provide improved means for reconstituting wet and dry medicament components.

### BRIEF SUMMARY OF THE DISCLOSURE

In accordance with the present invention there is provided an autoinjector comprising:
a first chamber for containing a dry component of a medicament;
a second chamber for containing a wet component of a medicament;
an axially-slidable stopper intermediate said first and second chambers;
an injection needle in fluid communication with said first chamber; and
a transfer needle initially disposed axially forward of said stopper, the transfer needle having a longitudinal axial bore therethrough, a closed forward or proximal end, an open rear or distal end and a radial aperture intermediate said proximal and distal ends and in fluid communication with said longitudinal axial bore;
wherein said transfer needle is capable of penetrating said stopper to establish fluid communication between said first and second chambers via said bore and radial aperture to enable said wet component to mix with said dry component and wherein said injection needle is capable of delivering the mixed dry and wet components of the medicament to an injection site.

Other features are described in the appended claims.

### BRIEF DESCRIPTION OF THE DRAWINGS

Preferred embodiments of the present invention will now be more particularly described, by way of example only, with reference to the accompanying drawings in which:

FIG. 1 is a cross sectional side view of the autoinjector device, prior to use, with a front end cap in place;

FIG. 2 is a partial cross sectional view of the autoinjector showing a detailed view of the transfer needle and front portion of the medicament cartridge;

FIG. 3 is a cross sectional side view of the device of FIG. 1 with the front end cap, front cap clip and needle shield removed;

FIG. 4a is a partial cross sectional view of the rear of the autoinjector of FIG. 1 prior to use;

FIG. 4b is a partial cross sectional view of the rear of the autoinjector of FIG. 4a immediately after the outer housing has been moved axially forward with respect to the reconstitution housing;

FIG. 4c is a partial cross sectional side view of the rear of the autoinjector of FIG. 4b immediately after the flexible hooks of the reconstitution ram have flexed radially inwards;

FIG. 4d is a partial cross sectional side view of the rear of the autoinjector of FIG. 4c where the reconstitution ram is free to advance axially forward with respect to the cartridge;

FIG. 5 is a cross sectional side view of the autoinjector device of FIG. 4d after the reconstitution ram has driven the cartridge and needle axially forward to deliver medicament;

FIG. 6a is a cross sectional side view of the autoinjector device of FIG. 5 immediately after the transfer needle has punctured the first stopper;

FIG. 6b is a partial cross section showing a detailed view of the transfer needle and first stopper of FIG. 6a;

FIG. 7 is a cross sectional side view of the autoinjector device of FIG. 6a when the second stopper has met the first stopper;

FIG. 8 is a cross sectional side view of the autoinjector of FIG. 7 after the first stopper and second stopper have travelled to the forward end of the cartridge and all transferable medicament has been delivered; and

FIG. 9 is a simplified cross sectional side view of an alternative embodiment of the autoinjector device, having a dual-purpose needle which is shown ready to deliver medicament.

### DETAILED DESCRIPTION

Throughout this application, the words “comprise” and “contain” and variations of the words, for example “comprising” and “comprises”, means “including but not limited to”, and is not intended to (and does not) exclude other components, integers or steps.

Throughout this application, the singular encompasses the plural unless the context otherwise requires. In particular, where the indefinite article is used, the specification is to be understood as contemplating plurality as well as singularity, unless the context requires otherwise.

Throughout this application, reference to a “forward” direction means the direction which is towards the patient when the injection device is in use. The “forward” or “proximal” end of the injection device is the end nearest the patient's skin when the device is in use. Similarly, reference to a “rearward” direction means the direction which is away from the patient and the “rearward” or “distal” end of the device is the end furthest from the patient's skin when the injection device is in use.

Throughout this application, reference to a “wet component of the medicament” means any liquid medicament, solvent, diluent, gel or other substantially liquid component. “Wet” does not imply a complete absence of any solid matter.

Throughout this application, reference to a “dry component of the medicament” means any solid, powder or other substantially dry component. “Dry” does not imply a complete absence of liquid, and the dry component may comprise a stiff paste or slurry for example.

Throughout this application, references to “reconstitution” include the term “mixing” wherein components of a medicament are mixed together with no solution, reaction or other chemical process necessarily taking place.

Features, integers, characteristics or groups described in conjunction with a particular aspect, embodiment or example of the invention are to be understood to be applicable to any other aspect, embodiment or example described herein unless incompatible therewith.

In general, an autoinjector includes a needle which is located within the housing of the device. Upon activation of a force-generating source, a portion of the needle extends out of the housing and penetrates the outer layer of skin to deliver medicament. An improved autoinjector is described in our international patent application, published under number WO 2005/070481. This device requires that the needle is moved axially so that it can appear beyond the end of the nozzle for the duration of the injection, after which the needle retracts automatically, so that it is never in sight of the user. The device also requires that the plunger is moved axially so that medicament is ejected. The overall complexity of the autoinjector is significantly reduced by both of these requirements being effected by one component, namely an inner housing (having reference numeral 7 in WO 2005/070481). Whilst it is preferable that the autoinjector described herein comprises a mechanism whereby the needle extends from and retracts into the nozzle automatically, this is not an essential feature.

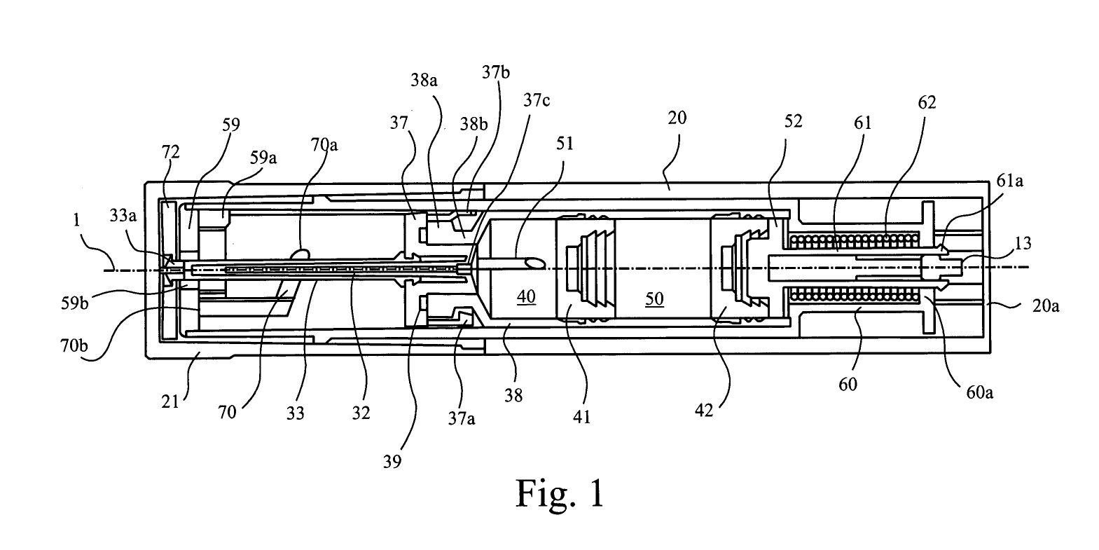

Referring to FIG. 1 of the present application, selected components of the autoinjector will now be described. The device has an outer housing 20 with a front end cap 21 on a front end thereof. Within the outer housing 20 and front end cap 21 there is a reconstitution housing 60 that contains a glass medicament cartridge 38. The cartridge 38 in isolation is open at both ends and has a generally cylindrical body with a narrow neck region 38b at a front end. The forwardmost part of the neck region 38b has a flange 38a having a larger diameter than the neck region 38b.

A plastic moulding 37 is located in the neck region 38b of the cartridge 38 and forms a seal around its front opening. The plastic moulding 37 is affixed to the flange 38a of the cartridge 38 by several hooks 37a located around the circumference of the plastic moulding 37. The seal is enhanced by an O-ring seal 39 located intermediate the flange 38a and the plastic moulding 37. At least two tags 37b extend radially outwards from the plastic moulding 37 and locate in apertures (not shown) in the reconstitution housing 60 thereby fixing the location of the plastic moulding 37 (and therefore cartridge 38) relative the reconstitution housing 60.

When assembled, the cartridge 38 has a dry chamber 40 defined as the volume within the cartridge 38 intermediate the plastic moulding and a first stopper 41. The dry chamber is for storage of the dry component of medicament.

At the rear of the cartridge 38 a second stopper 42 is located axially rearward of the first stopper 41. A wet chamber is then defined as the volume within the cartridge 38 intermediate the first stopper 41 and second stopper 42. Both the first stopper 41 and second stopper 42 are slidably located within the cartridge 38 and can move along an axial path within the cartridge 38 whilst maintaining a seal. Thus the first stopper 41 provides a seal between the dry chamber 40 and wet chamber 50 and normally does not permit mixing of the dry and wet medicament components.

Embedded in plastic moulding 37 are two needles, namely an injection needle 32 and a transfer needle 51. The injection needle 32 extends forwardly from the plastic moulding 37 and is preferably coaxial with a central longitudinal axis 1 of the autoinjector device. The transfer needle 51 extends rearwardly from the plastic moulding 37 into the cartridge 38 and is offset radially from the central longitudinal axis 1 of the device. The transfer needle 51 is therefore also offset radially from the injection needle 32, provided that the injection needle 32 is coaxial with central longitudinal axis 1.

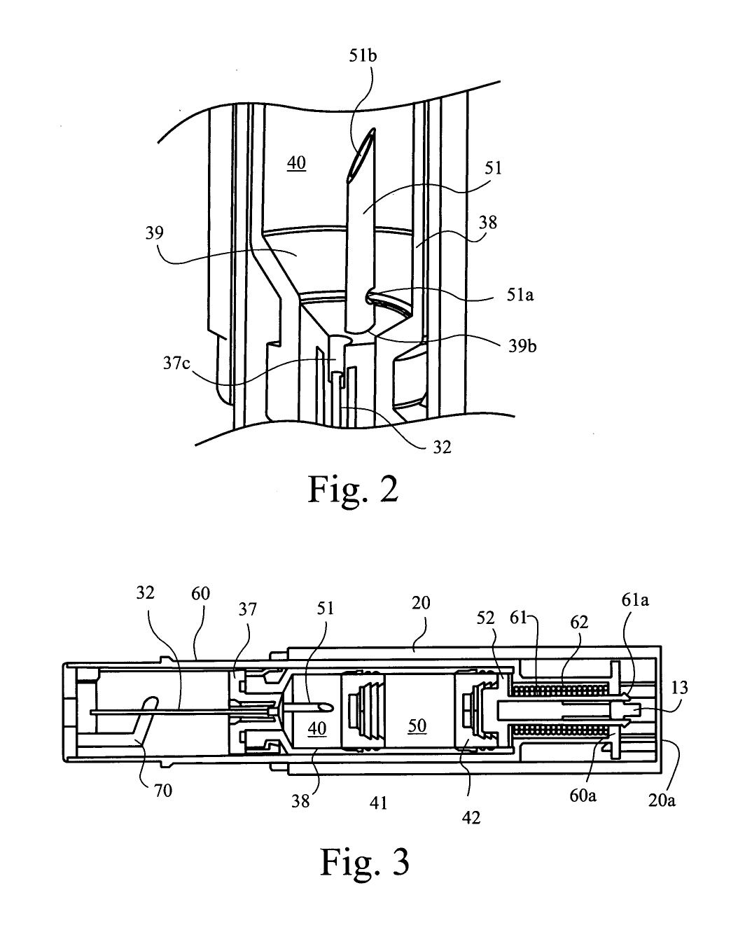

The injection needle 32 is embedded in an open hole 37c within the plastic moulding 37 such that the injection needle 32 is in fluid communication with the dry chamber 40 (see FIG. 2 for example). Conversely, the transfer needle 51 is embedded in a “blind hole” 39b within the plastic moulding 37.

As can be seen from FIG. 2, the transfer needle 51 has an axial bore 51b therethrough. The transfer needle 51 is open at its rearmost end so that the bore 51b is in fluid communication with the dry chamber 40 (prior to reconstitution), and is closed at its forwardmost end within the blind hole 39b. The transfer needle 51 is provided with a radial hole 51a at a point axially forward from the open rear end which extends the bore 51b in a radial direction. The radial hole is also in fluid communication with the dry chamber 40.

When assembling the device, the wet and dry medicament components must be installed into the cartridge 38. The dry component is first placed in the front end of the cartridge 38 around the transfer needle 51. Once the dry medicament has been placed in the cartridge 38, the first stopper 41 can be installed, ensuring an air space remains in the dry chamber 40. The air space permits the first stopper 41 to move forwards during actuation such that the transfer needle 51 can pierce it, as will be described further below.

The first stopper 41 forms a seal between the dry chamber 40 and the remainder of the cartridge 38 such that the liquid medicament component can then be filled into the cartridge 38 without entering the dry chamber 40. Once the liquid component has been filled into the cartridge 38, the second stopper 42 is installed. At this point, the cartridge 38 and the medicaments contained therein are sealed at a front end by the plastic moulding 37 and at a rear end by the second stopper 42. It is therefore important that conditions remain sterile at least until the second stopper 42 is installed. The forward end of the dry chamber 40 is in fluid communication with injection needle 32 that is embedded in plastic moulding 37. Sterile conditions are maintained by a needle cover 33 that surrounds injection needle 32, fitting into moulding 37 at a rear end and sealing the dry chamber 40. The needle cover 32 is described further below.

Once the medicament components have been loaded into the cartridge 38 and sealed therein, the cartridge 38 is installed into the reconstitution housing 60. FIG. 1 shows the autoinjector with the cartridge 38 installed prior to reconstitution.

At the front end of the reconstitution housing 60 is a front housing 59 connected thereto by hooks 59a. The front housing 59 has an aperture 59b located centrally on the central longitudinal axis 1 through which the needle 32 can protrude when an injection is delivered. Surrounding the front housing 59 and a front portion of the reconstitution housing 60 is a front cap clip 72. The front cap clip 72 has outwardly extending hooks (not shown) that locate in apertures (not shown) of the front end cap 21 affixing the front cap clip 72 thereto.

For the user to use the device and deliver medicament, the front end cap 21 and front cap clip 72 must first be removed. Removal of the front end cap 21 exposes part of the reconstitution housing 60 since it projects from within the outer housing 20. To remove the front end cap 21 a forward axial force must be applied to overcome the hooks of the front cap clip 72.

The reconstitution housing 60 has at least one guide channel 70 therein which may be an aperture or a groove or the like. The guide channel 70 is preferably helical and is illustrated in FIG. 1 where it can be seen that the guide channel has a closed end 70a and an open end 70b. The front housing 59 also has at least one groove (not shown) that forms a continuation of the open end 70b of the at least one guide channel 70.

The guide channel 70 is adapted to receive at least one guide pin (not shown) projecting radially inwardly from an inner surface of the front cap clip 72. Prior to use, the front end cap fits over the reconstitution housing 60 and the guide pin is disposed in the guide channel 70 and axial movement of the front cap clip 72 relative the reconstitution housing 60 is prevented. This feature prevents the front cap clip 72 from being removed when the front end cap 21 is pulled axially forward.

It is possible to twist the front cap clip 72 relative the reconstitution housing 60, but only in one direction as the guide pin is initially disposed adjacent the closed end 70a of the guide channel 70. When the front cap clip 72 is twisted relative to the reconstitution housing 60, the guide pin travels along the guide channel 70 so that the reconstitution housing 60 (and remainder of the device) moves axially relative to the front cap clip 72. Thus, when the guide pin reaches the open end 70b of the guide channel 70, the front cap clip 72 can be removed entirely from the device (the guide pin passing through the groove(s) in the front housing).

In alternative embodiments, the front cap may be removed by pulling along a straight axial path. However, the helical guide channel arrangement described above is preferable as it prevents independent movement of the internal components of the device, which may lead to unintentional firing, for example if the device was dropped.

Prior to use, the needle shield 33 surrounds the needle 32 thus protecting it from damage, ensuring it remains sterile, and preventing any potential injury to the user. The forwardmost end of the needle shield 33 extends through an aperture located on the central axis of the front cap clip 72. A flanged end 33a of the needle shield 33 having a larger diameter than the aperture in the front cap clip 72 is disposed on a front side of the front cap clip 72. Removal of the front cap clip 72 therefore causes the removal of the needle shield 33 also. In one preferable embodiment, a quarter of a turn is sufficient to remove the front cap clip 72 and the needle shield 33. Once the front end cap 21, front cap clip 72 and needle shield 33 have been removed, as shown in FIG. 3, the device is ready for use.

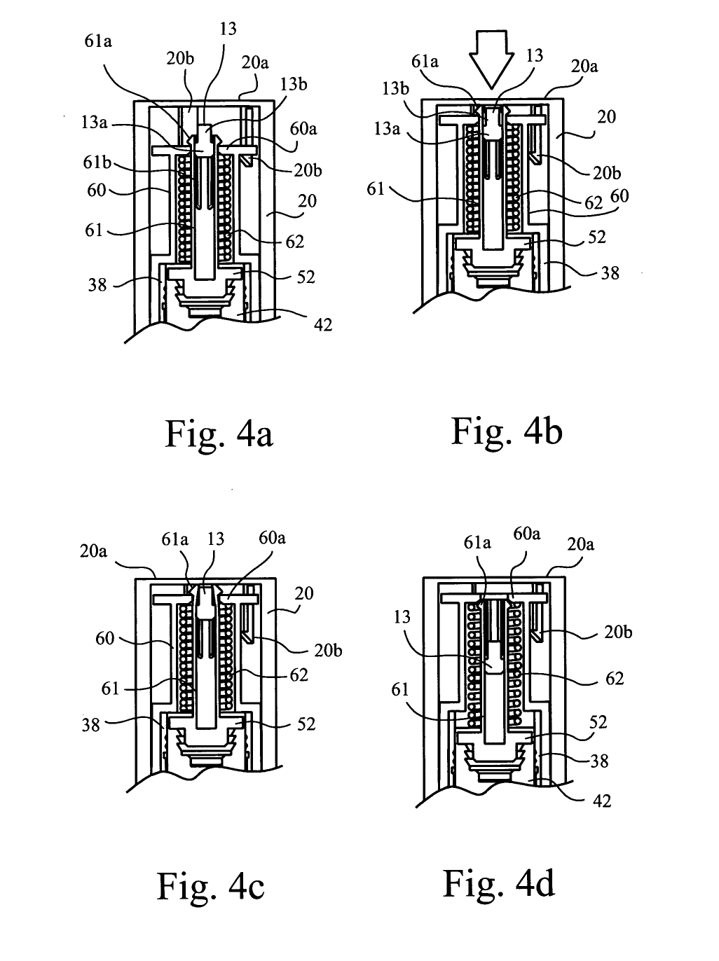

The front of the device is then placed against an injection site and the outer sleeve 20 is pushed axially forward by the user to begin reconstitution. The mechanism by which reconstitution begins will be described with reference to FIGS. 4a-4d.

FIG. 4a shows a cross sectional view of the rear of the device before the outer sleeve 20 is pushed axially forward. From FIG. 4a, it can be seen that the outer housing 20 has several hooks 20b that extends axially forward from an inner surface of a rear end 20a. When in the condition illustrated by FIG. 4a, the hooks 20b prevent forward axial movement of the reconstitution housing 60 relative the outer housing 20, thus retaining the reconstitution housing 60 within the outer housing 20.

As shown in FIG. 4a, the reconstitution housing 60 has a narrow rear section 60a through which protrudes a reconstitution ram 61 (prior to use). The rear of the reconstitution ram 61 is generally cylindrical and has a plurality of flexible hooks 61a defined by axial slots 61b where the flexible hooks extend radially outwards to a diameter greater than that of the narrow section 60a. Thus, the flexible hooks 61a prevent forward axial movement of the reconstitution ram 61 relative the reconstitution housing 60. Before reconstitution has been actuated by the user pushing the outer sleeve 20 axially forward, a reconstitution pin 13 is disposed within the flexible hooks 61a (as shown in FIG. 4a).

The reconstitution ram 61 extends axially forwards and connects to a reconstitution stopper 52 at a front end. Surrounding the reconstitution ram 61 within the reconstitution housing 60 is a spring 62. In FIG. 4a, the spring 62 is shown to be under compression acting rearwardly against the rear of the reconstitution housing 60 and forwardly against reconstitution stopper 52.

The reconstitution pin 13 has a wide section 13a disposed axially forward of a narrow section 13b. Both the wide section 13a and the narrow section 13b are generally cylindrical where diameter of the wide section 13a is larger than that of the narrow section 13b. When the outer housing 20 is pushed axially forwards (indicated by arrow in FIG. 4b), the rear of the outer housing 20 abuts the reconstitution pin 13 also causing it to move axially forwards to the position shown in FIG. 4b. When the wide section of the reconstitution pin 13 has passed the narrow section 60a at the rear of the reconstitution housing (as shown in FIG. 4b) the flexible hooks 61a are free to flex radially inwards so that their axial path is no longer blocked by the narrow section 60a of the reconstitution housing 60 (FIG. 4c). The flexible hooks 61 a may have a chamfered leading edge to assist them in flexing radially inwards. Since the flexible hooks 61a (when flexed radially inwards) no longer prevent forward axial movement of the reconstitution ram 61 relative the reconstitution housing 60, the reconstitution ram 61 is free to move axially forwards under the influence of the spring 62.

The frontmost edge of the forwardly advancing outer sleeve 20 causes the tags 37b of the plastic moulding 37 located in apertures (not shown) of the reconstitution housing 60 to dislocate from their apertures thereby permitting movement of the plastic moulding 37 (and therefore cartridge 38) relative the reconstitution housing 60.

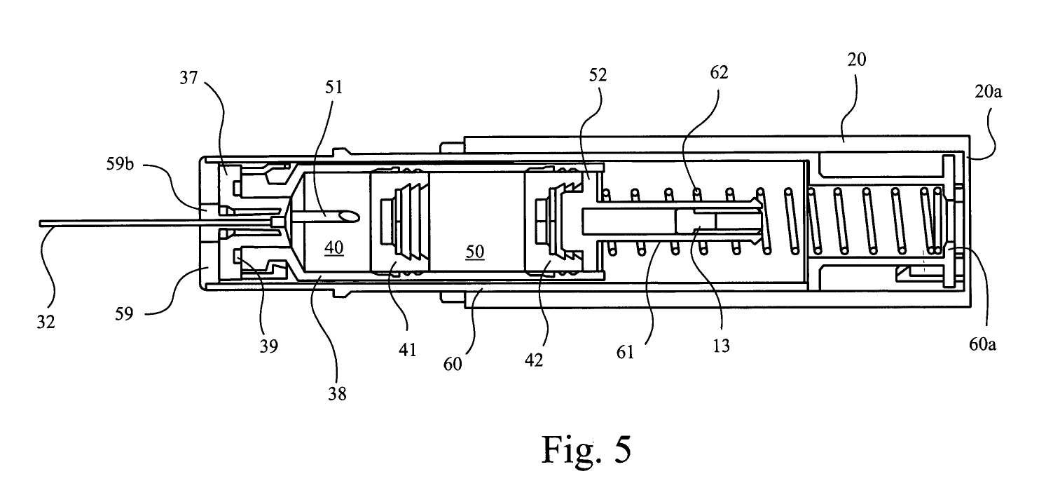

The forwardly advancing reconstitution ram 61 forces the reconstitution stopper 52 to advance forwards which abuts the second stopper 42 causing it too to move axially forwards. Due to the force of spring 62, the advancing reconstitution ram 61 causes the cartridge 38, plastic moulding 37 and injection needle 32 to move axially forwards also. As the cartridge 38 travels to the forward most end of the reconstitution housing 60, the plastic moulding 37 abuts the front housing 59 and the injection needle 32 projects through the aperture 59b of the front housing 59 to penetrate an injection site (FIG. 5). The needle is now ready to deliver medicament.

As a result of the plastic moulding 37 being in abutment with the front housing 59, the cartridge 38, plastic moulding 37, and injection needle 32 cannot move axially forward any further. Since the spring 62 has not yet been fully decompressed, the elastic spring force continues to act through the reconstitution ram 61. This causes the reconstitution stopper 52 and second stopper 42 to collectively move axially forward relative to the cartridge 38. Due to the incompressible nature of the liquid medicament contained within the wet chamber 50, the forward axial movement of the second stopper 52 drives the first stopper 41 axially forward a substantially identical distance. The first stopper 41 therefore moves axially forward relative to the cartridge 38 and is pierced by the transfer needle 51. The above mentioned air space in the dry chamber 40 permits forward axial movement of the first stopper 41 relative the cartridge 38. Without an air space, the first stopper 41 would not move axially forward relative the cartridge 38 due to the substantially incompressible dry medicament therein.

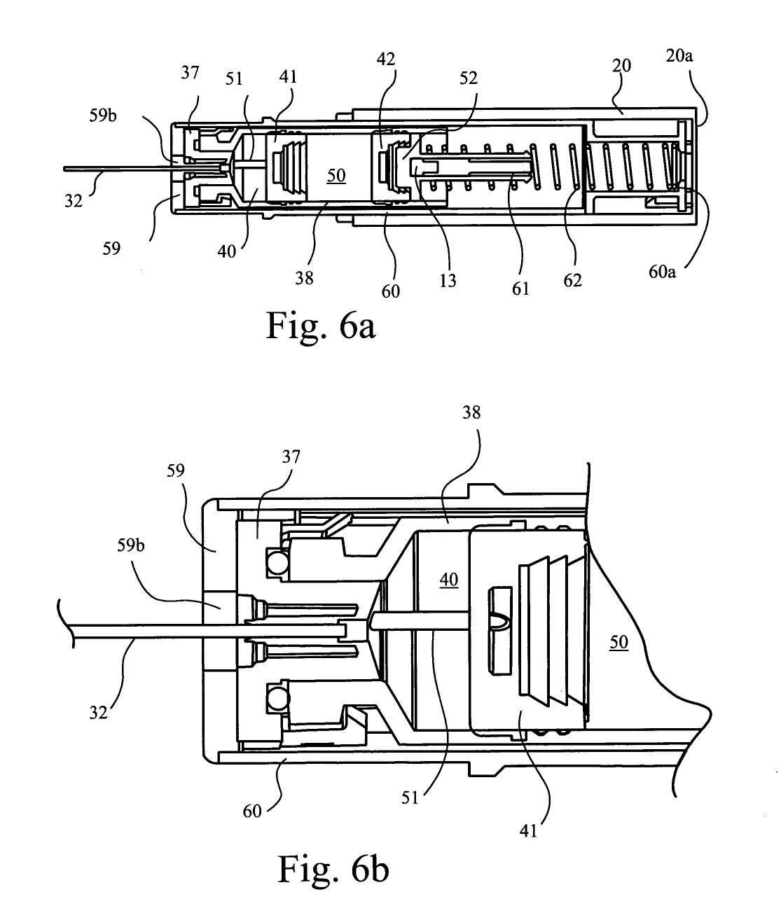

As the transfer needle penetrates the first stopper 41, fluid communication between the wet chamber 50 and the dry chamber 40 is established via the bore 51b and radial hole 51 a of the transfer needle 51. FIG. 6a shows the device immediately after fluid communication between the wet chamber 50 and the dry chamber 40 has been established. FIG. 6b provides a more detailed view of the transfer needle 51 and first stopper 41 as shown in FIG. 6a. The liquid medicament component then flows from the wet chamber 50 into the dry chamber 40 and mixes with dry medicament component before exiting the dry chamber 40 through the injection needle 32. This process is known in the art as “flush through” mixing. Depending on the solubility of the dry medicament, the liquid exiting the injection needle will be a solution. In an alternative embodiment, a filter may be disposed in the open hole 37c of the plastic moulding 37 to prevent solid matter from entering the injection needle 32 and potentially causing a blockage. Use of a filter may also aid mixing as solid medicament would be held on the filter surface whilst the liquid medicament flushes through.

The rate of flow from the wet chamber 50 to the dry chamber 40 is limited by the dimensions of the transfer needle 51. In particular, the rate of flow can be controlled from a design perspective by altering the diameter of the transfer needle 51 (and its bore 51b), and the size and position of the radial hole 51a. In an alternative embodiment, the transfer needle 51 may comprise one or more holes that are not necessarily radial holes, through which the wet medicament component can flow from the wet chamber 50 to the dry chamber 40.

In a further embodiment, the dry chamber 40 may include baffles that manage the flow of liquid medicament and enhance mixing of the wet and dry components.

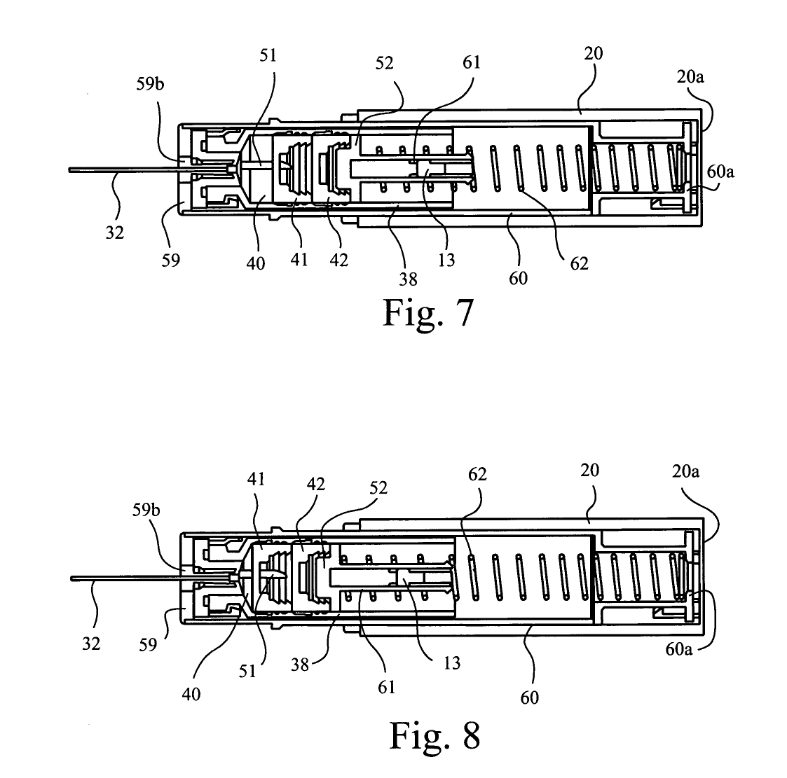

Liquid medicament will continue to flow from the wet chamber 50 to the dry chamber 40 through transfer needle 51 until the second stopper 42 meets the first stopper 41. FIG. 7 shows the device immediately after the second stopper 42 has made contact with the first stopper 41. At this point, the liquid medicament has been expelled from the wet chamber 50. It should be noted that, owing to the geometry of the first and second stoppers 41, 42, some liquid medicament may remain in the wet chamber 50. The spring 62 continues to act axially forward pushing the reconstitution ram 61, reconstitution stopper 52, second stopper 42 and first stopper 41 collectively forward relative the cartridge 38. As this happens, the first stopper 41 slides over the transfer needle 51 until it cannot move axially forward any further with the cartridge 38. This action expels the remaining transferable mixed medicament from the dry chamber 40 through the injection needle 32 and the injection is completed.

During injection, the dry chamber 40 is vented through the injection needle 32, so the air space in the dry chamber 40 also permits some flow of liquid medicament between the particles of the dry medicament.

FIG. 8 shows the device when the first stopper 41 has been pushed to its forwardmost position with the cartridge 38. It will be noted from FIG. 8 that due to the shape of the cartridge 38, the dry chamber 40 will not be reduced to a zero volume when the first stopper 41 is in its forwardmost position within the cartridge 38. This is important with regards to the position of the radial hole 51a. While the radial hole 51a may be located anywhere on the transfer needle 51, it is preferable that it is located such that it is not blocked by the advancing first stopper 41 at any stage during its axial movement. Thus, a preferable location for the radial hole 51a is on a part of the transfer needle 51 that always remains within the dry chamber 40 (and not embedded in the first stopper 41) when the first stopper 41 has advanced to the front of the cartridge 38.

Since the dry chamber 40 is not reduced to a zero volume when the first stopper 41 has travelled its full forward distance within the cartridge 38, some medicament may remain within the dry chamber 40 after the injection process is complete. However, this can be compensated for by careful selection of the quantities of medicaments initially loaded into the device.

In alternative embodiments, means may be provided for automatically retracting the needle 32 into the reconstitution housing after medicament has been delivered, or after a suitable dwell time. In other embodiments, the needle 32 may also be driven forward by driving means to deliver an injection such that pressurisation of the liquid medicament component in the wet chamber 50 would not necessarily be required.

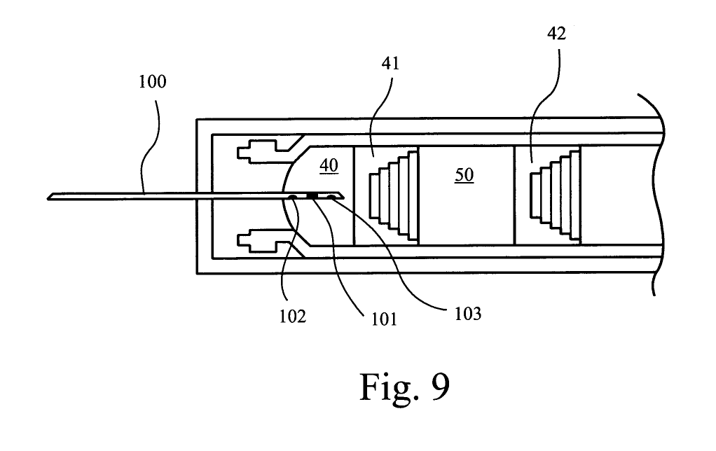

In a further embodiment shown in FIG. 9, one needle is provided which serves the functions of both the transfer needle 51 and the injection needle 32. Such a dual-purpose needle may comprise a double-ended needle 101 having a crimp or other blockage 101 part-way down and a radial hole 102, 103 on each side of the blockage. FIG. 9 shows the device in a comparable position to the device shown in FIG. 5, i.e. with the needle 100 ready to deliver an injection but with the medicament not yet mixed. The forwardmost radial hole 102 is equivalent to the open rear end of needle 32 in FIG. 5 and allows the needle 100 to be in fluid communication with the dry chamber 40. The rearmost radial hole 103 is equivalent to the radial hole 51a described above. The blockage 101 is equivalent to the closed end of transfer needle 51 described above. The relative spacing of the blockage 101 and radial holes 102, 103 in relation to the stopper 41 allows the dual-purpose needle 100 to function in the same manner as both the injection needle 32 and transfer needle 51 described above.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. An autoinjector comprising:
a first chamber for containing a dry component of a medicament;
a second chamber for containing a wet component of a medicament;
an axially-slidable stopper intermediate said first and second chambers;
an injection needle in fluid communication with said first chamber; and
a transfer needle initially disposed axially forward of said stopper, the transfer needle having a longitudinal axial bore therethrough, a closed forward or proximal end, an open rear or distal end and a radial aperture intermediate said proximal and distal ends and in fluid communication with said longitudinal axial bore;
wherein said transfer needle is capable of penetrating said stopper to establish fluid communication between said first and second chambers via said bore and radial aperture to enable said wet component to mix with said dry component and wherein said injection needle is capable of delivering the mixed dry and wet components of the medicament to an injection site.

2. The autoinjector of claim 1 wherein said injection needle is radially offset relative to said transfer needle.

3. The autoinjector of claim 1 wherein said transfer needle has a larger diameter than said injection needle.

4. The autoinjector of claim 1 wherein said first and second chambers are part of a medicament cartridge.

5. The autoinjector of claim 4 wherein said medicament cartridge includes a moulding to seal its forward end.

6. The autoinjector of claim 1 further comprising an outer housing with a reconstitution housing therein and a front end cap.

7. The autoinjector of claim 6, wherein said first and second chambers are part of a medicament cartridge, and wherein said medicament cartridge is located within said reconstitution housing.

8. The autoinjector of claim 6 wherein one of said reconstitution housing and said front end cap is provided with a guide channel, and the other of said reconstitution housing and said front end cap is provided with a guide pin for engaging in said guide channel so that, in use, relative movement between said front end cap and reconstitution housing is guided by the travel of said pin in said groove.

9. The autoinjector of claim 6 wherein forward movement of said outer housing relative to said reconstitution housing is capable of initiating mixing of the wet and dry components of the medicament.

10. The autoinjector of claim 1 wherein the first and/or the second chambers contain one or more baffles for controlling the flow of medicament therein.

11. The autoinjector of any of claim 6 wherein, after delivery of the injection, the injection needle is capable of retracting into said housing.

12. The autoinjector of claim 4 wherein the distal end of said cartridge is sealed with a second axially-slideable stopper.

13. The autoinjector of claim 1 wherein said injection needle and said transfer needle are coaxial.

14. The autoinjector of claim 1 wherein said injection needle is coaxial with a central longitudinal axis of the autoinjector.

15. The autoinjector of claim 14 wherein said dual-purpose needle comprises two radial apertures with a blockage or crimp therebetween.

16. (canceled)

17. The autoinjector of claim 4 wherein said medicament cartridge is a glass medicament cartridge.

18. The autoinjector of claim 5 wherein said moulding is an O-ring.

19. The autoinjector of claim 8 wherein said guide channel comprises a helical groove.

20. The autoinjector of claim 13 wherein said injection needle and said transfer needle comprise a single dual-purpose needle.

21. The autoinjector of claim 1 further comprising a dry component of a medicament disposed in the first chamber and a wet component of a medicament disposed in the second chamber.
